(19) United States
(12) Patent Application Publication
(10) Pub. No.: US 2008/0241288 A1
(43) Pub. Date: Oct. 2, 2008

(54) Method and composition of a carminative herb or natural supplement to decrease the adverse events of orlistat, an oral lipase inhibitor

(76) Inventor: Ronald J. Thompson, Fort Thomas, KY (US)

Correspondence Address: Donald N. Halgren, 35 Central Street, Manchester, MA, 01944-1311, US

(21) Appl. No.: 12/070,391

(22) Filed: Feb. 15, 2008

Related U.S. Application Data
(62) Division of application No. 11/585,633, filed on Oct. 24, 2006, now abandoned.
Application No. 11/585,633 is a continuation-in-part of application No. 11/522,627, filed on Sep. 18, 2006, now abandoned.

(51) Int. Cl. A61K 36/38; A61K 36/534; A61K 36/288; A61P 39/00; A61K 36/906; A61K 36/886

(52) U.S. Cl. 424/730; 424/747; 424/764; 424/756; 424/744

(57) ABSTRACT

A medicament to decrease the adverse events of Orlistat, an oral lipase inhibitor, arranged to be taken in combination with Orlistat, comprising: an admixture of carminative herbs and one or more chelating agents to absorb and mechanically agitate non-digested fats.

This invention relates to medicaments and more particularly to herbs and nutritional supplements for use in weight loss programs and further for improvements in the use of a pharmaceutical known as Orlistat, and is a continuation-in-part application of my co-pending U.S. patent application Ser. No. 11/522,627, filed 18 Sep. 2006, entitled “Method and Composition of a Medicament to Decrease the Adverse Effects of Orlistat, an Oral Lipase Inhibitor, incorporated herein by reference in its entirety.

### FIELD OF THE INVENTION

1. Background of the Invention and Prior Art

Orlistat is the generic name for Xenical® (Roche), a prescription lipase inhibitor, FDA approved for weight loss and obesity management. Lipase is the pancreatic enzyme that breaks down ingested fats into small chain fatty acids in the lower lumen of the stomach, and the small intestine. By inhibiting the actions of the lipase enzyme, ingested fats cannot be absorbed, and weight loss naturally occurs. The problem with Olistat treatment is, the most undesirable side effects are caused by the passage of the undigested fats through the gastrointestinal tract! The Physicians Desk Reference lists the adverse events (side effects) of Olistat clinical trials on over 2800 patients for one or two years as:

Upper Gastrointestinal Adverse Events

| | |
|---|---|
| Abdominal pain/discomfort | 25.5% |
| Nausea | 8.1% |

Lower Gastrointestinal Adverse Events

| | |
|---|---|
| Oily spotting | 26.6% |
| Flatus - with discharge | 23.9% |
| Fecal urgency | 22.1% |
| Fatty/oily stool | 26.0% |
| Oily evacuation | 11.9% |
| Increased defecation | 10.8% |
| Fecal incontinence | 7.7% |

Orlistat, as a lipase inhibitor, creates a mal-absorption state, where ingested fats are not absorbed by the intestine, and therefore must be eliminated through the lower intestine and rectum. All of the adverse events are directly caused by the elimination of large sized fat globules.

2. Prior Art

The chemical name for Orlistat is tetrahydrolipstatin. U.S. Pat. No. 4,598,089 issued Jul. 1, 1986, incorporated herein by reference in its entirety, defines tetrahydrolipstatin, and teaches its unique lipase inhibitor actions. These actions are further defined in U.S. Pat. Nos. 5,245,056 and 5,399,720, (both incorporated herein by reference), to treat obesity and various medical conditions associated with obesity, specifically diabetes and hypertension. U.S. Pat. No. 6,696,467, (incorporated herein by reference) further teaches and defines the specific benefits of the lipase inhibitor tetrahydrolipstatin for the treatment of obesity by weight reduction and appetite suppression. U.S. Pat. No. 6,004,996 (incorporated herein by reference), describes the production of tetahydrolipstatin into microspheres for optimal therapeutic delivery into the lumen of the stomach. These microspheres have a very efficient action as a lipase inhibitor, because of the large surface area to bind to the lipase enzyme.

### DESCRIPTION OF THE DRAWINGS

The objects and advantages of the present invention will become more apparent when viewed in conjunction with the following drawings, in which:

FIG. 1 represents an outline of a stomach and intestine showing a Lipase enzyme released and breaking down fat;

FIG. 2 represents the outline of FIG. 1 showing Olistat blocking the Lipase enzyme to prevent the absorption of fat; and

FIG. 3, represents the outline of FIG. 2, with a chelate admixed therewith, which decreases the surface tension of the fat to prevent the formation of large fat globules.

### DETAILED DESCRIPTION OF THE PRESENT INVENTION

The present invention comprises a medicament arrangement to decrease the adverse effects caused by the pharmaceutical agent Orlistat in weight loss therapy. A carminative herb is an herb or nutritional supplement that is utilized to improve digestion or to treat dyspepsia or irritable bowel symptoms of ulcerative colitis. These carminative herbs, usually in a combination with other carminative herbs have heretofore been used to improve digestion and to treat maladies of specific organs in the digestive system, such as gall bladder, pancreas, liver, stomach or intestines both large and small. Carminative herbs however, have not been employed or utilized to treat the side effects or adverse events of specific weight loss medications. Such surprising results when used in combination with the use of the weight loss drug Orlistat, offers the consumer an opportunity to avoid such problems realized when Orlistat is used by itself.

A list of “carminative” herbs, used as herbs or admixed with other herbs to prepare an herbal or nutritional supplement medicament is:
Mentha Spicata (peppermint)
Zingiber Offinate (ginger)
Foeniculum Vulgare (fennel)
Pimpinella Anisum (anise)
Melissa Officinalis (lemon balm)
Taraxacum Officinale (dandelion)
Aloe Vera (aloe)
Iberis Amara (candy tuft)
Valeriana Officinaus (valerian)
Carbo Vegetabilius
Nux Vomica
Chamomilla
Fennel Seed
Caraway Seed
Clove Oil
Wormwood Oil
Dill Oil
Turmeric Oil
Barberry
Bitter Orange
Blessed Thistle
Cardamom
Centaury
Chicory
Cinnamon
Coriander
Denil's Claw
Gentian
Horehound
Juniper
Linden
Milk Thistle
Radish
Rosemary
Sage
St John's Wort
Varrom
Capsaicin
Artichoke Leaf Extract
Boldo
Cayenne
Banana Powder

It is certain that several of the carminative herbs have a surfactant-like action to emulsify fats. The emulsification of fats to prevent the formation of large globules is the method to treat or even prevent some of the listed adverse events of Orlistat therapy.

The earlier referenced U.S. patent application Ser. No. 11/522,627 entitled “Method and Composition of Medicament to Decrease the Adverse Events of Orlistat, an Oral Lipase Inhibitor” uses the carminative herb peppermint, to provide generalized bowel relaxation in addition to specific emulsifier simethicone and chelating/mechanical agitator agents, activated charcoal and methyl cellulose.

2. Chelating Agents as Mechanical Activators

Once in a small enough physical state by the actions of the emulsifying agent, the chelating agent will absorb the undigested unabsorbed fat from the stomach and small intestine to decrease Orlistat related adverse events. The hallmark chelating agent in treatment of toxic poisoning is activated carbon (activated charcoal). Activated carbon is not absorbed, but remains in the gastrointestinal tract and eliminated with bowel movements. The activated carbon will chelate free fats in the gastrointestinal lumens. A second chelating agent is cellulose or methylcellulose, a non-fermentable fiber that will, like activated carbon, chelate the small emulsified fats that result from orlistat therapy. Any chelating agent could be utilized, but for example, activated carbon and methylcellulose are the most effective and are thus the preferred substances here.

A second mode of action of the chelating agents, activated charcoal, is to physically and mechanically decrease the surface tension of undigested fats. This is a mechanism which have the inert granules and methylcellulose fibers are kept in a churning motion in the small intestine by the inherent peristalsis of the circular smooth muscles of the small intestines. This churning motion mechanically disrupts the formation of large fat globules. The two mechanisms to decrease surface tension, chemical and mechanical, synergistically function to prevent the formation of large fat globules, the pathogenesis of the adverse effects of Orlistat.

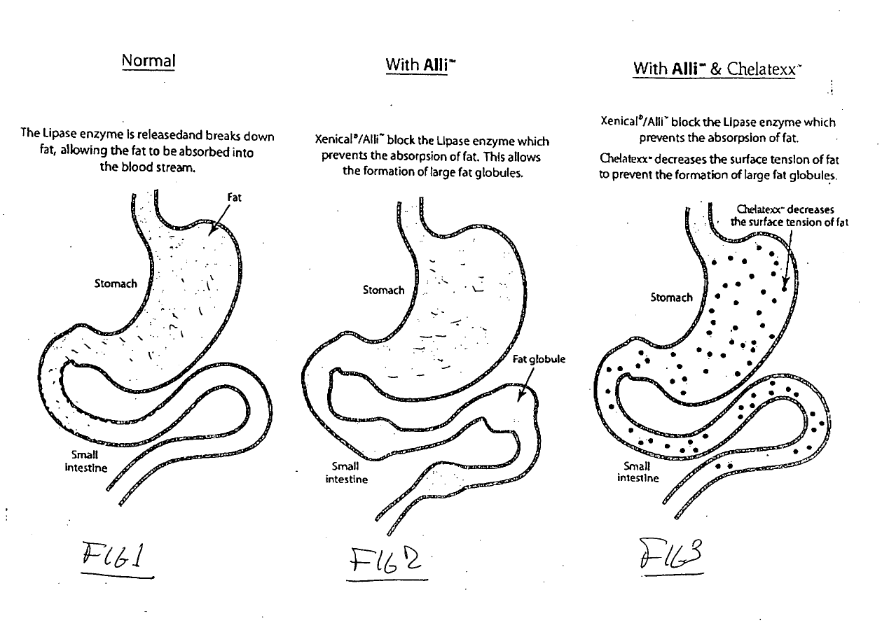

The present invention is also represented through the FIGS. 1, 2 and 3. In FIG. 1, the Lipase enzyme is released and breaks down the fat in the stomach, allowing that fat to be absorbed in the bloodstream in a normal manner. In FIG. 2, Olistat, the chemical name for products such as Xenical® and Alli™ are shown blocking the Lipase enzyme which prevents the absorption of fat. This also allows the formation of large fat globules. FIG. 3 represents the blocking of the Lipase enzyme by Xenical or Alli, preventing the absorption of fat, with the present invention, a carminative herb admixture and chelating agent/mechanical agitator preventing the formation of large fat globules.

The invention thus comprises a medicament to decrease the adverse events of Olistat, an oral lipase inhibitor, in combination with Olistat, comprising: a carminative herb; a surfactant, and one or more chelating agents to absorb non-digested fats. The surfactant preferably comprises for example, at least one of the following: Simethicone, Stearoxy dimethicone, Dimethicone, Methicone, Amino bispropyl dimethicine, Amino propyl dimethicine, Amodimethicone, Amodimethicine hydroxysterate, Dehyroxy dimethricine, C24-28 alkyl methicine, C30-45 alkyl methicine, C30-45 alkyl dimethicine, Certearyl methicine and Cetyl Dimethicine. The carminative agent preferably comprises for example, at least one of the following: Mentha Spicata (peppermint), Zingiber Offinate (ginger), Foeni-culum Vulgare (fennel), Pimpinella Anisum (anise), Melissa Officinalis (lemon balm), Taraxacum Officinale (dandelion), Aloe Vera (aloe), Iberis Amara (candy tuft), Valeriana Officinaus (valerian), Carbo Vegetabilius, Nux Vomica, Chamomilla, Fennel Seed, Caraway Seed, Clove Oil, and Wormwood Oil. The carminative agent preferably comprises for example, at least one of the following: Dill Oil, Turmeric Oil, Barberry, Bitter Orange, Blessed Thistle, Cardamom, Centaury, Chicory, Cinnamon, Coriander, Denil's Claw, Gentian, Horehound, Juniper, Linden, Milk Thistle, Radish, Rosemary, Sage, St John's Wort, Varrom, Capsaicin, Artichoke Leaf Extract, Boldo, Cayenne or Banana Powder. The chelating agents preferably comprise at least one of the following: Peppermint, Activated Carbon and Methyl cellulose. The chelating agents are for example, taken in the following ranges, said Peppermint 90 mg/capsule 50-200 mg; said Activated Carbon 300 mg/capsule 100-500 mg, and said Methyl cellulose 200 mg/capsule 100-400 mg,

The invention also comprises a medicament to decrease the adverse events of Olistat, an oral lipase inhibitor, in combination with Olistat, comprising for example: a carminative agent comprising at least one of: Mentha Spicata (peppermint), Zingiber Offinate (ginger); Foeniculum Vulgare (fennel); Pimpinella Anisum (anise), Melissa Officinalis (lemon balm), Taraxacum Officinale (dandelion), Aloe Vera (aloe), Iberis Amara (candy tuft), Valeriana Officinaus (valerian), Carbo Vegetabilius, Nux Vomica, Chamomilla, Fennel Seed, Caraway Seed, Clove Oil, Wormwood Oil, and an emulsifier comprised of at least one of the following: Simethicone, Stearoxy dimethicone, Dimethicone, Methicone, Amino bispropyl dimethicine, Amino propyl dimethicine, Amodimethicone, Amodimethicine hydroxysterate, Dehyroxy dimethricine, C24-28 alkyl methicine, C30-45 alkyl methicine, C30-45 alkyl dimethicine, Certearyl methicine and Cetyl Dimethicine; and one or more chelating agents to absorb non-digested fats, wherein said chelating agent comprises at least one of the following: Activated Carbon and Methyl cellulose.

The invention also comprises a method to decrease the adverse events of Olistat, an oral lipase inhibitor, comprising one or more of the following steps: taking orally Olistat and a compound of an emulsifier, a carminative agent and at least one chelating agent to absorb non-digested fats; and mechanically agitate said fats by said chelating agent. The agent is preferably for example, one or more of the following: Simethicine, Peppermint, Activated Carbon and Methyl cellulose. Olistat is tetrahydrolipstatin. The carminative agent is selected for example from one or more of the following: Mentha Spicata (peppermint), Zingiber Offinate (ginger); Foeniculum Vulgare (fennel); Pimpinella Anisum (anise), Melissa Officinalis, (lemon balm), Taraxacum Officinale (dandelion), Aloe Vera (aloe), Iberis Amara, (candy tuft), Valeriana Officinaus (valerian), Carbo Vegetabilius, Nux Vomica, Chamomilla, Fennel Seed, Caraway Seed, Clove Oil and Wormwood Oil. The carminative agent comprises for example, at least one of the following: Dill Oil, Turmeric Oil, Barberry, Bitter Orange, Blessed Thistle, Cardamom, Centaury, Chicory, Cinnamon, Coriander, Denil's Claw, Gentian, Horehound, Juniper, Linden, Milk Thistle, Radish, Rosemary, Sage, St John's Wort, Varrom, Capsaicin, Artichoke Leaf Extract, Boldo, Cayenne and Banana Powder.

1.-7. (canceled)

8. A method to decrease the adverse events of Olistat, an oral lipase inhibitor, comprising:
taking orally Olistat and a compound of an emulsifier, a carminative agent and at least one chelating agent to absorb non-digested fats; and
mechanically agitate said fats by said chelating agent.

9. The method as recited in claim 8, wherein said agent is one or more
of the following: Simethicine, Peppermint, Activated Carbon and Methyl cellulose

10. The method of claim 8, wherein Olistat is tetrahydrolipstatin.

11. The method of claim 8, wherein said carminative agent is selected from one or more of the following: Mentha Spicata (peppermint), Zingiber Offinate (ginger); Foeniculum Vulgare (fennel); Pimpinella Anisum (anise), Melissa Officinalis (lemon balm), Taraxacum Officinale (dandelion), Aloe Vera (aloe), Iberis Amara (candy tuft), Valeriana Officinaus (valerian), Carbo Vegetabilius, Nux Vomica, Chamomilla, Fennel Seed, Caraway Seed, Clove Oil and Wormwood Oil.

12. The method of claim 8, wherein said carminative agent comprises at least one of the following: Dill Oil, Turmeric Oil, Barberry, Bitter Orange, Blessed Thistle, Cardamom, Centaury, Chicory, Cinnamon, Coriander, Denil's Claw, Gentian, Horehound, Juniper, Linden, Milk Thistle, Radish, Rosemary, Sage, St John's Wort, Varrom, Capsaicin, Artichoke Leaf Extract, Boldo, Cayenne and Banana Powder.
